(19) United States
(12) Patent Application Publication
(10) Pub. No.: US 2020/0022908 A1
(43) Pub. Date: Jan. 23, 2020

(54) METHOD FOR TREATING MYOPIA AND APPLICATION IN PREPARATION OF MEDICAMENT

(71) Applicant: WENZHOU MEDICAL UNIVERSITY, Wenzhour City, Zhejiang (CN)

(72) Inventors: Xiangtian ZHOU, Wenzhou City, Zhejiang (CN); Sen ZHANG, Wenzhou City, Zhejiang (CN); Jia QU, Wenzhou City, Zhejiang (CN); Guoyun ZHANG, Wenzhou City, Zhejiang (CN); Fei ZHAO, Wenzhou City, Zhejiang (CN); Xuan ZHOU, Wenzhou City, Zhejiang (CN)

(21) Appl. No.: 16/486,785

(22) PCT Filed: Mar. 22, 2019
(86) PCT No.: PCT/CN2019/079184

§ 371 Date: Aug. 16, 2019

(30) Foreign Application Priority Data
Jul. 18, 2018 (CN) 2018107875411

(51) Int. Cl. A61K 9/00; A61P 27/02

(52) U.S. Cl. CPC A61K 9/0048; A61K 9/0019; A61P 27/02

(57) ABSTRACT

A method for treating myopia and an application in preparation of a medicament are provided, where the volume of choroidal blood flow is increased by expanding the choroid via a drug or a surgical method, so as to achieve treatment of the myopia caused by scleral hypoxia, which provides a new idea and method for treating myopia.

### RELATED APPLICATIONS

The present application is a National Phase entry of PCT Application No. PCT/CN2019/079184, filed Mar. 22, 2019, which claims priority to Chinese Patent Application No. 2018107875411, filed Jul. 18, 2018, the disclosures of which are hereby incorporated by reference herein in their entirety.

### FIELD OF THE INVENTION

The present invention relates to the technical field of myopia treatment, and in particular to a method for treating myopia, and an application in preparation of a medicament.

### BACKGROUND

A choroid plays an important role in refractive development and myopia occurrence. It has been found through a large number of animal experiments that the choroidal thickness is thinned when the myopia is formed. Similar results are observed in human population, and the choroidal thickness of a hypermetropic human population is significantly thicker than that of each of emmetropic and myopic human populations. Our research team has found that, the thinning of the choroidal thickness occurs in addition to changing of an ocular anterior segment when human eyes are adjusted for looking the close, but the mechanism of this change is not well understood.

A sclera is a final effector for the occurrence and development of myopia, where after the myopia occurs, collagen fibers of the sclera become thinner and have a disordered arrangement, the scleral collagen is reduced, and the sclera is thinned. However, its regulatory mechanisms and trigger factors are still unclear. There are many types of cells in the sclera. In the past research, the whole scleral tissue is taken as a research object, such that the information derived from different cell sources is mixed and cannot accurately reveal cell-specific changes, and thus it is difficult to accurately explain the regulation mechanism of collagen synthesis in a scleral fibroblast. Our team has found that expression of a scleral hypoxia-inducible factor 1α (H IF-1α) is up-regulated when the myopia occurs, and returned back to normal when the myopia is recovered, suggesting that the scleral tissue is in an anoxic state when the myopia occurs.

Hypoxia of the scleral tissue may be a cause for extracellular matrix remodeling and myopia. The sclera belongs to a connective tissue, which itself has fewer blood vessels, and a large part of oxygen is derived from choroidal vessels. The choroid is mainly composed of a vascular network, so changing of its thickness is likely to result from regulation of choroidal blood flow. The function of the choroid is mainly supplying nutrients and oxygen to a retina and participating in regulation of scleral growth.

### SUMMARY

In order to remedy the deficiencies of the prior art, an objective of the present invention is to provide a method for treating myopia and an application in preparation of a medicament, where a myopia caused by scleral hypoxia is treated by increasing the volume of choroidal blood flow.

The technical solutions adopted by the present invention are: a method for treating myopia, where the method is treating the myopia caused by scleral hypoxia by increasing the volume of choroidal blood flow,

where the method is increasing the volume of choroidal blood flow by expanding the choroid, so as to achieve treatment of the myopia caused by the scleral hypoxia,

the method is increasing the volume of choroidal blood flow by expanding the choroid via a medicine, so as to achieve treatment of the myopia caused by the scleral hypoxia;

application of a drug for increasing the volume of choroidal blood flow in preparation of a medicament for treating myopia,

where the drug for increasing the volume of choroidal blood flow is a drug that directly dilates a blood vessel or vascular smooth muscle or a drug that indirectly dilates a blood vessel.

The drug that directly dilates a blood vessel or vascular smooth muscle is one or more of nicotinic acid, hydralazine, sodium nitroprusside, indapamide, dibazole, papaverine, cinnarizine, merislon or nitroglycerin.

The drug that indirectly dilates a blood vessel is one or more of a histamine drug, alpha-adrenaline, an angiotensin converting enzyme inhibitor drug, a calcium channel blocker, an anticholinergic agent that blocks a M cholinergic receptor or a traditional Chinese medicine that improves blood circulation.

The histamine drug is merislon.

The α-adrenergic receptor blocker is one or more of phentolamine, hydergine or prazosin.

The angiotensin converting enzyme inhibitor drug is captopril.

The calcium channel blocker is one or more of nifedipine or verapamil.

The anticholinergic agent that blocks a M cholinergic receptor is anisodamine.

The traditional Chinese medicine that improves blood circulation is one or more of ligusticum wallichii, salviae miltiorrhizae, radix pueraiae, a Weierkang tablet, a Chuanshen capsule or Shuxuening.

The beneficial effects of the present invention are: The present invention provides a method for treating myopia and an application in preparation of a medicament, where the volume of choroidal blood flow is increased by expanding the choroid via a drug or a surgical method, so as to achieve treatment of the myopia caused by scleral hypoxia, which provides a new idea and method for treating myopia.

### BRIEF DESCRIPTION OF THE DRAWINGS

FIG. 1 shows the change of the scleral hypoxia-inducible factor 1α (HIF-1α) when myopia occurs; NC-L: Normal control left eye, NC-R: Normal control right eyed-F: Form-deprived fellow eye, FD-T: Form-deprived test eye, I-F: Minus-lens induced fellow eye, I-T: Minus-lens induced test eye, Rec-F: Recovered fellow eye, Rec-T: Recovered test eye.

FIG. 2 is a diagram that shows the choroidal thickness and blood flow of spontaneous myopia and hyperopia; FIG. 2A: diagram of choroid thickness, FIG. 2B: diagram of choroidal blood flow, FIG. 2C: correlation between choroidal thickness and choroidal blood flow. Control: a normal group, Myopia: a spontaneous myopia group, Choroidal Thickness (ChT): choroidal thickness, Chorodal blood flow (ChBF): volume of choroidal blood flow.

FIG. 3 is a diagram showing various parameters for 7 days of form deprivation and a recovery period: FIG. 3A: a diagram showing the difference of binocular refractions, FIG. 3B: a diagram showing the difference of binocular axial lengths, FIG. 3C: a diagram showing the difference of binocular choroidal thicknesses, FIG. 3D: a diagram showing the difference of binocular choroidal blood flows, FIG. 3E: Correlation between choroidal thickness and choroidal blood flow. Base: Before the test, Control7D: 7 days of a normal group, FD7D: 7 days of formal deprivation, Control11D: 11 days of a normal group, R-FD: 4 days after form deprivation, Choroidal Thickness (ChT): choroidal thickness, Chorodal blood flow (ChBF): choroidal blood flow.

FIG. 4 is a diagram showing various parameters for 7 days of lens-induction and a recovery period: FIG. 4A: a diagram showing the difference of binocular refractions, FIG. 4B: a diagram showing the difference of binocular axial lengths, FIG. 4C: a diagram showing the difference of binocular choroidal thicknesses, FIG. 4D: a diagram showing the difference of binocular choroidal blood flows, FIG. 4E: Correlation between choroidal thickness and choroidal blood flow. Base: Before the test, Plano7D: 7 days of plano lens, LIM7D: 7 days of minus lens, R-Plano: 4 days with the plano lens removed, R-LIM: 4 days after the minus lens is removed, Choroidal Thickness (ChT): Choroidal thickness, Chorodal blood flow (ChBF): Choroidal blood flow.

FIG. 5 is a schematic diagram showing the results for the effect of the drug (nicotinic acid) that directly dilates a blood vessel or vascular smooth muscle on form deprivation: Where FIG. 5A: a diagram showing the difference of binocular refractions, FIG. 5B: a diagram showing the difference of binocular axial lengths, FIG. 5C: a diagram showing binocular choroidal blood flows, FIG. 5D: Correlation between choroidal thickness and choroidal blood flow. Vehicle: Solvent, NA: nicotinic acid, ChT: choroidal thickness, Chorodal blood flow (ChBF): choroidal blood flow, ChBF Signal Points: signal points in the choroidal blood flow.

FIG. 6 is schematic diagram showing the results for the effect of the α-adrenergic receptor blocker (α-blocker: prazosin) on form deprivation: FIG. 6A: a diagram showing the difference of binocular refractions, FIG. 6B: a diagram showing the difference of binocular axial lengths, FIG. 6C: a diagram showing binocular choroidal blood flows, FIG. 6D: Correlation between choroidal thickness and choroidal blood flow. Vehicle: Solvent, Pr: prazosin, ChT: choroidal thickness, Chorodal blood flow (ChBF: choroidal blood flow, ChBF Signal Points: signal points in the choroidal blood flow.

### DESCRIPTION OF THE EMBODIMENTS

In order to explain the present invention more clearly, the specific embodiments are described below, and the specific embodiments do not limit the scope of the present invention.

Hypoxia of the scleral tissue may be a cause for extracellular matrix remodeling and myopia. The sclera belongs to a connective tissue, which itself has fewer blood vessels, and a large part of oxygen is derived from choroidal vessels. The choroid is mainly composed of a vascular network, so changing of its thickness is likely to result from regulation of choroidal blood flow. The function of the choroid is mainly supplying nutrients and oxygen to a retina and participating in regulation of scleral growth. Therefore, we have experimentally proved that the decrease of the volume of choroidal blood flow causes scleral hypoxia, thereby leading to myopia.

### Experimental Operations

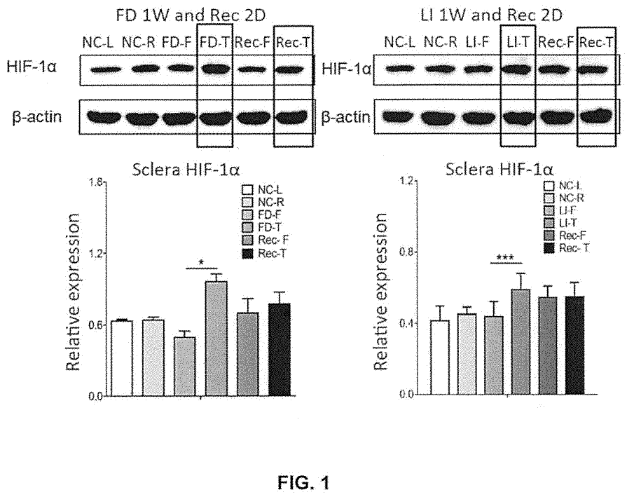

(1) We conducted form deprivation or lens induction of 3 week-aged guinea pigs, and found that expression of a scleral hypoxia-inducible factor 1α (HIF-1α) in a test eye is up-regulated when the myopia occurs, and returned back to normal when the myopia is recovered (FIG. 1), suggesting that the scleral tissue is in an anoxic state when the myopia occurs.

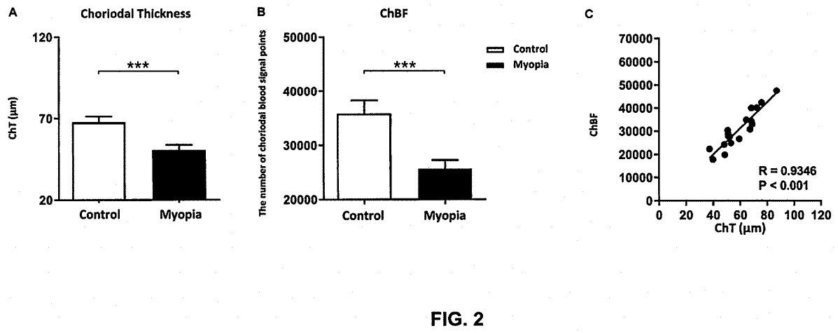

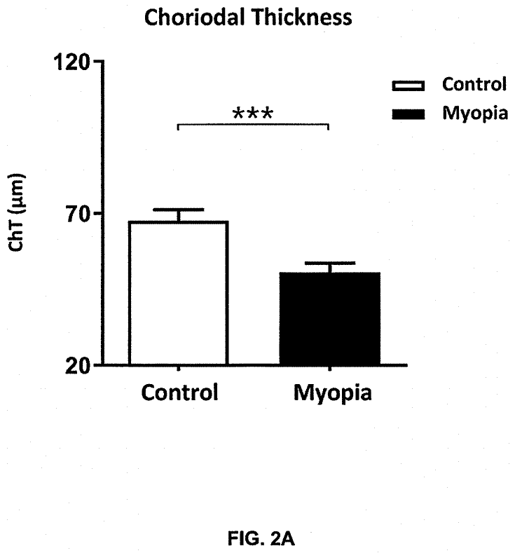

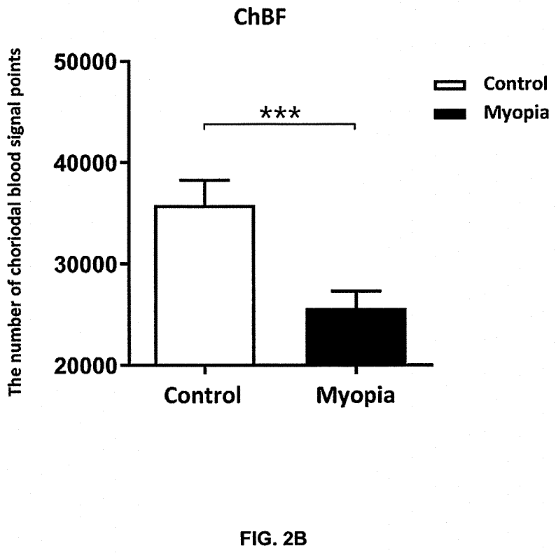

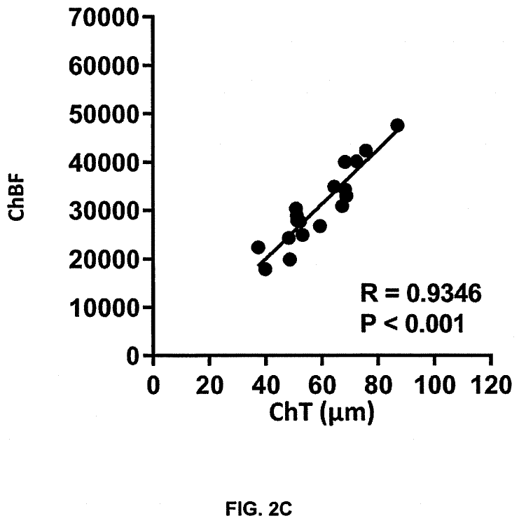

(2) Through detection of the choroids of 3-week-aged spontaneous myopia guinea pigs (refractive power: −5.94±1.28 Diopter) and normal guinea pigs (refractive power: +3.36±0.80 Diopter), we found that the choroidal thickness is significantly thinned and the blood flow is significantly reduced in the spontaneous myopia guinea pigs as compared with the normal guinea pigs. (normal group vs. spontaneous myopia group: choroid thickness: 67.65±3.58 μm vs. 50.60±3.14 μm, P<0.01; choroidal blood flow: 35825.89±2445.03 vs. 25633.22±1665.09, P<0.01). Also there is a high correlation between the choroidal thickness and the choroidal blood flow (R=0.9346, p<0.001), as shown in detail in FIG. 2A-2C.

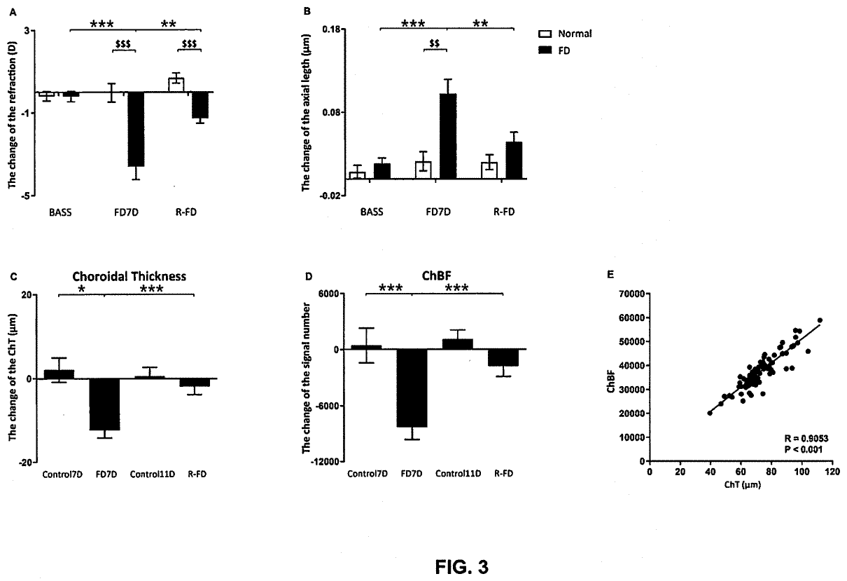

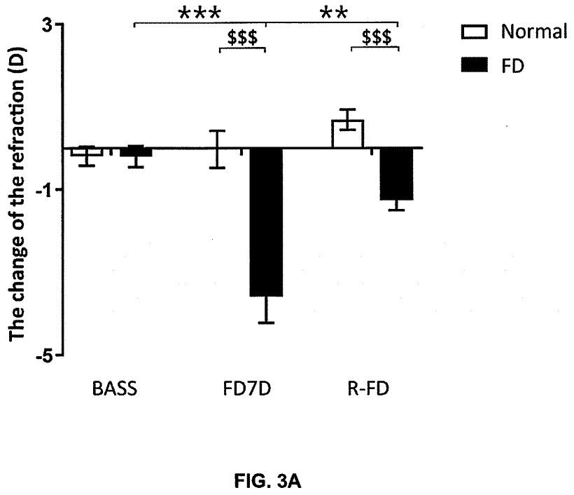

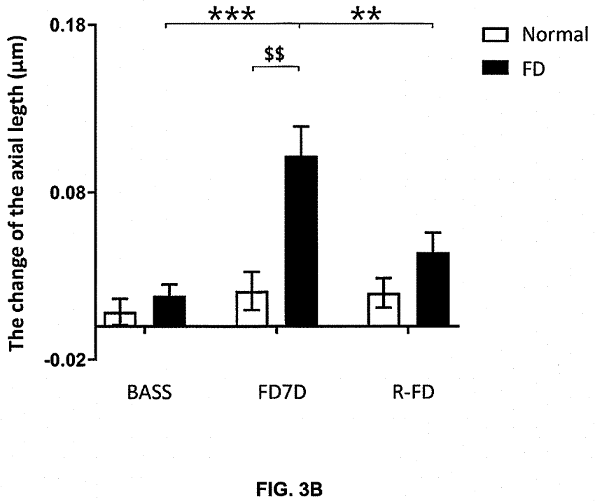

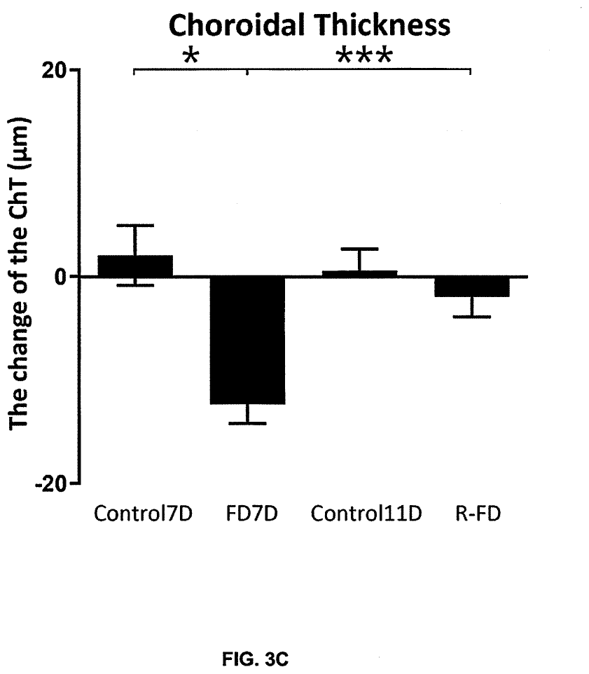

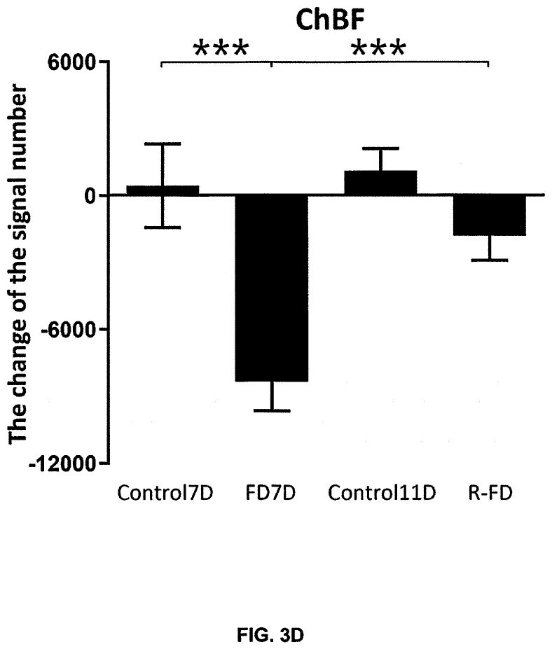

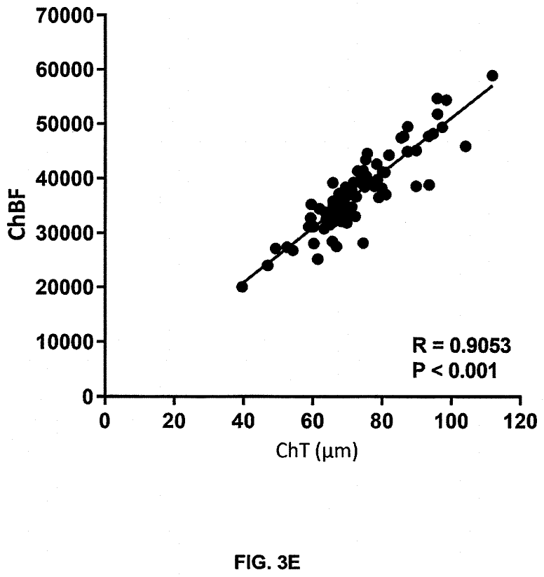

(3) We conducted form deprivation for 7 days on the 3 week-aged guinea pigs (Form deprivation, FD) to produce an obvious myopia, and the myopia was significantly recovered 4 days after the deprivation factors were removed. (Base vs. FD 7D: 5.46±0.65 D vs. 1.00±1.13 D, p<0.01; FD vs. R-FD: 1.00±1.13 D vs. 2.73±0.63 D, p<0.05), and the eye axis has a corresponding response. The choroidal thickness is significantly thinned or the blood flow is significantly reduced on day FD7 and is statistically significant (normal group vs. FD group vs. R-FD: choroid thickness 69.8±13.2 μm vs. 64.3±11.3 μm vs. 77.0±12.1 μm; choroidal blood flow: 36548±6368 vs. 32448±5815 vs. 38706±6356), and there is a high correlation between the choroidal thickness and the choroidal blood flow (R=0.9053, p<0.001), as shown in detail in FIG. 3A-3E.

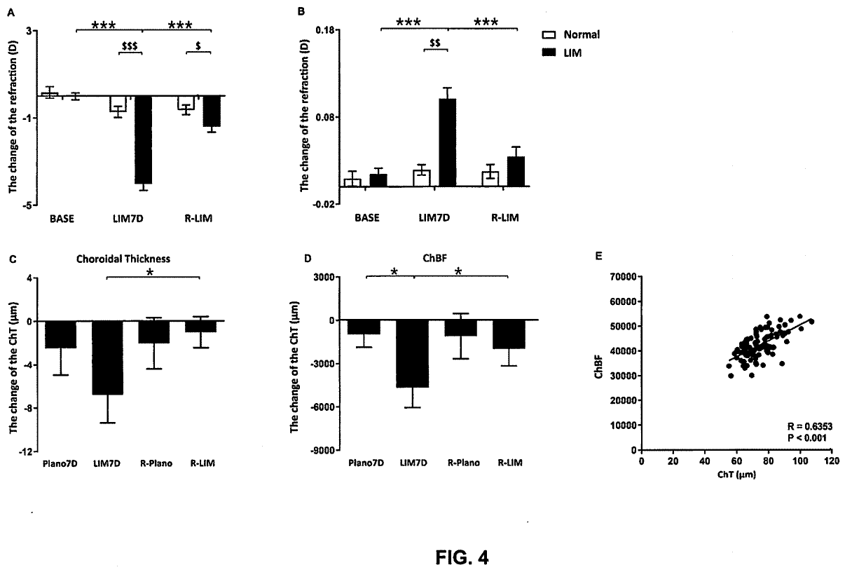

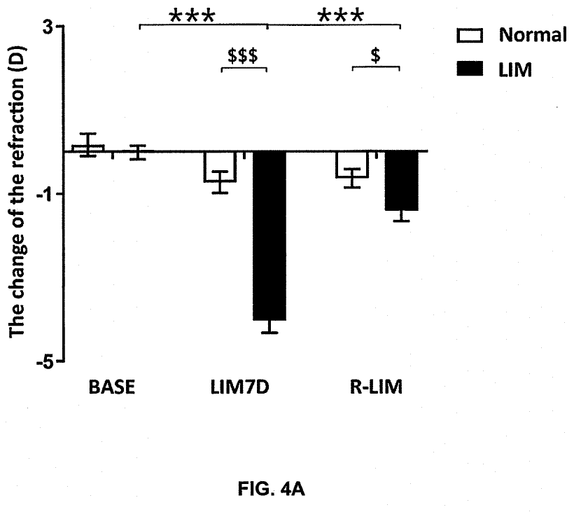

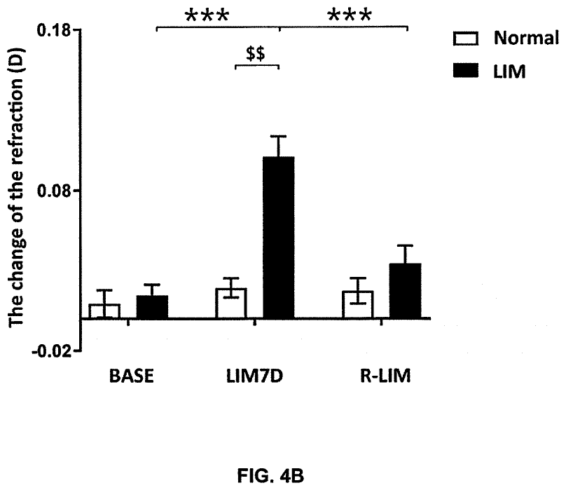

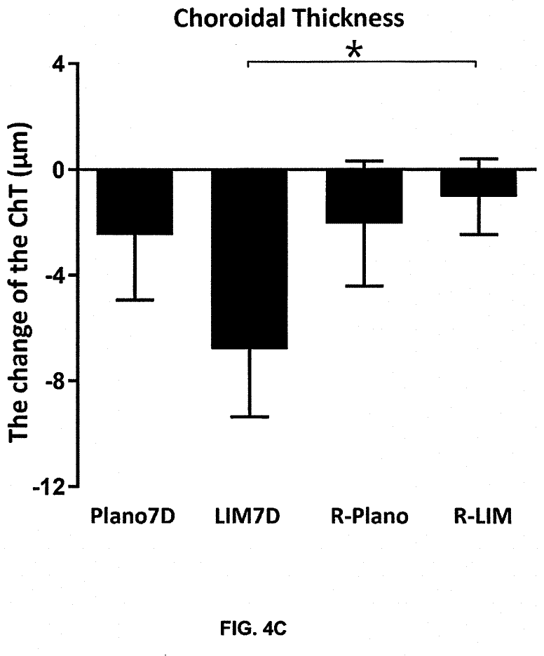

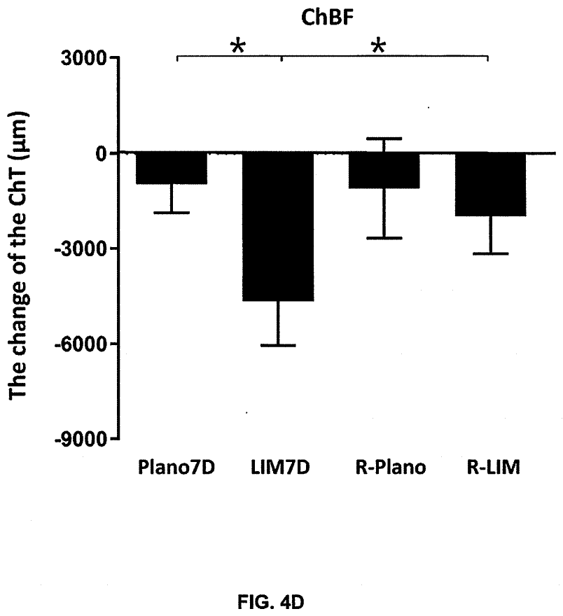

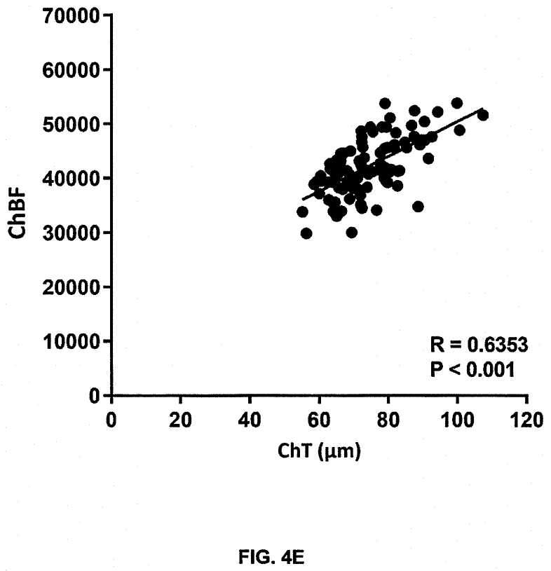

(4) We conducted lens-induction for 7 days on the 3 week-aged guinea pigs (Lens induced, LI) to produce an obvious myopia, and the myopia is significantly recovered 4 days after the induction factors are removed. (Base vs. LI 7D: 5.70±0.25 D vs. 1.11±0.59 D, p<0.001; LI 7D vs. R-LIM: 1.11±0.59 D vs. 3.54±0.43 D, p<0.001), and the eye axis has a corresponding response. The choroidal thickness is significantly thinned or the blood flow is significantly reduced on day LI7 and is statistically significant (normal group vs. LI group vs. R-LI: choroid thickness 73.1±10.0 μm vs. 68.2±7.9 μm vs. 74.6±8.6 μm; choroidal blood flow: 40887±5116 vs. 38975±3931 vs. 41965±4012), and there is a high correlation between the choroidal thickness and the choroidal blood flow (R=0.6353, p<0.001), as shown in detail in FIG. 4A-4E.

We found that the decrease of the volume of choroidal blood flow causes scleral hypoxia, thereby leading to myopia. Through the above experiments, we believe that myopia can be effectively controlled by increasing blood flow and increasing oxygen supply through expansion of the choroid.

Therefore, in the present invention, the myopia caused by scleral hypoxia is treated by increasing the volume of choroidal blood flow.

In the present invention, by expanding the choroid via a drug or a surgical method, the oxygen supply to the sclera is increased, thereby increasing the volume of choroidal blood flow to achieve treatment of the myopia caused by scleral hypoxia.

The present invention can effectively control myopia by increasing blood flow and increasing oxygen supply through expansion of the choroid via the following drugs:

1. a drug that directly dilates a blood vessel or vascular smooth muscle: nicotinic acid, hydralazine, sodium nitroprusside, indapamide, dibazole, papaverine, cinnarizine, merislon, nitroglycerin, and the like;

2. a histamine drug that has the effect of dilating capillaries and increasing cerebral blood flow: merislon (betahistine), etc.;

3. a α-adrenergic receptor blocker (α-blocker): phentolamine (phentolamine mesylate), hydergine, prazosin, etc.;

4. an angiotensin converting enzyme inhibitor drug: captopril (Clikelyopril), etc.;

5. a calcium channel blocker, such as nifedipine and verapamil;

6. an anticholinergic agent that blocks a M cholinergic receptor: anisodamine, etc.; and

7. a traditional Chinese medicine that improves blood circulation: ligusticum wallichii, salviae miltiorrhizae, radix pueraiae, a Weierkang tablet, a Chuanshen capsule, Shuxuening, and the like.

The mechanism is as follows:

1. The drug that expands the choroid is a drug that directly dilates a blood vessel or vascular smooth muscle.

Principle: it has a direct relaxation effect on the vascular smooth muscle, reduces peripheral resistance and has a vasodilating effect.

2. the drug that expands the choroid is a histamine drug that has the effect of dilating capillaries and can increase the blood flow.

Principle: the binding of histamine to a receptor (H1R) on a vascular smooth muscle causes vasodilation, increases the permeability of blood vessels, and leads to local increase in blood flow.

3. the drug that expands the choroid is a α-adrenergic receptor blocker.

Principle: an a-receptor blocker refers to a blocker that selectively binds to a α-adrenoceptor, does not agonize or attenuate agonization of adrenergic receptors, but can block the binding of corresponding neurotransmitters and drugs to the a-receptor to produce an anti-adrenergic effect. By effects of blocking the α1 receptor of the vascular smooth muscle and directly dilating the vascular smooth muscle, the blood vessels are dilated and the resistance is reduced.

One class is drugs that can compete for receptors with catecholamine to exert an effect of blocking the a-receptor, which work fast and act for a short duration due to the not very strong binding of them to the a-receptor, and are called short-acting a-receptor blockers. They are also known as competitive a-receptor blockers. Commonly used is phentolamine (Regitine). The other class is drugs that covalently bind to the a-receptor, have strong binding and features of having a strong receptor blocking action, long action time and the like, and are called long-acting class a-receptor blockers. They are also known as non-competitive α-receptor blockers, such as prazosin.

4. The drug that expands the choroid is an angiotensin converting enzyme inhibitor drug.

Principle: reducing generation of angiotensin II: inhibiting angiotensin converting enzymes enables reduction of generation of angiotensin II (effects of the angiotensin II: the angiotensin II binds to an angiotensin receptor to cause corresponding physiological effects including contraction of systemic arterioles and veins; increased transmitter release of sympathetic vasoconstrictor fibers; and promotion of endothelin release), and meanwhile it can also reduce the degradation of bradykinin, causes vasodilatation, and reduces the peripheral resistance.

5. The drug that expands the choroid is a calcium channel blocker.

Principle: inflow of extracellular Ca2+ is repressed to reduce the available intracellular Ca2+, and thus the effect is achieved. The blood vessels are dilated, the afterload is reduced, the release of certain important endogenous growth factors is reduced, or the growth-promoting effects of the following endogenous growth factors are antagonized: angiotensin II (its effects are as mentioned in 4), endothelin (endothelin is an endogenous long-acting vasoconstriction regulatory factor), catecholamine (the primary physiological effect of the catecholamine is exciting the α-receptor of the blood vessels to achieve vasoconstriction), and the like.

6. The drug that expands the choroid is an anticholinergic agent that blocks a M cholinergic receptor.

Principle: this drug binds to a M cholinergic receptor, and has effects of relaxing the smooth muscle, relieving vasospasm and improving microcirculation.

7. The drug that expands the choroid is a traditional Chinese medicine that improves blood circulation.

Now we verify the effect of treating the myopia caused by scleral hypoxia by increasing the volume of choroidal blood flow through expansion of the choroid via a drug that dilates the blood vessels. The results are as follows:

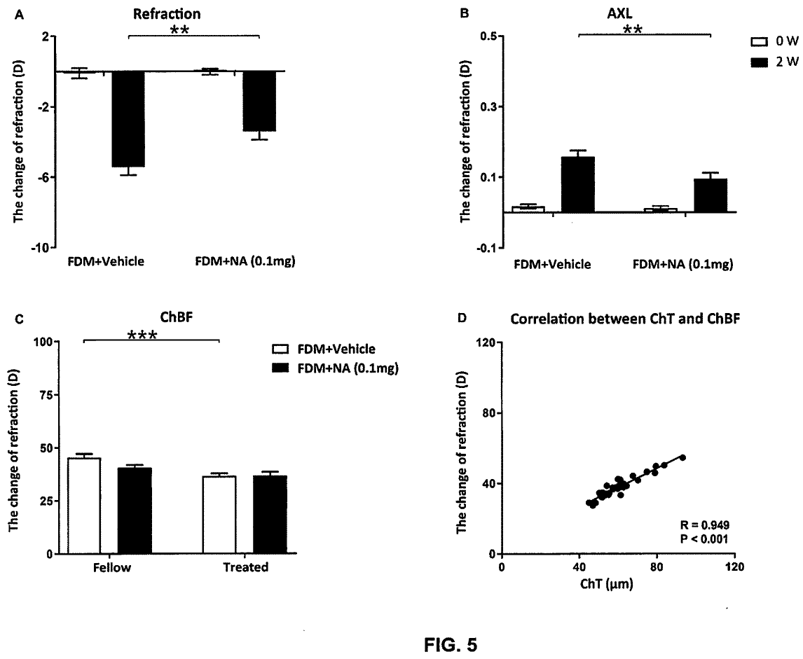

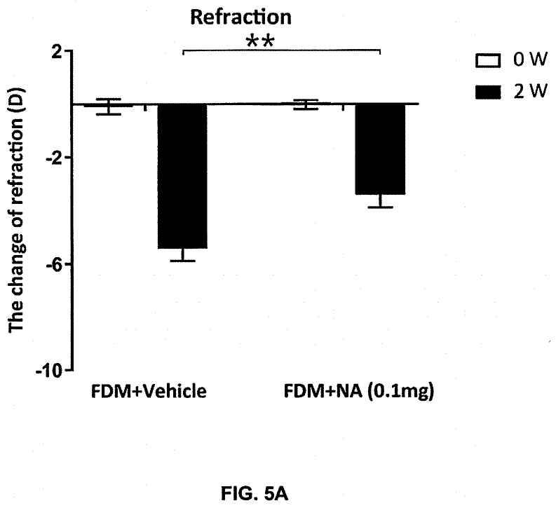

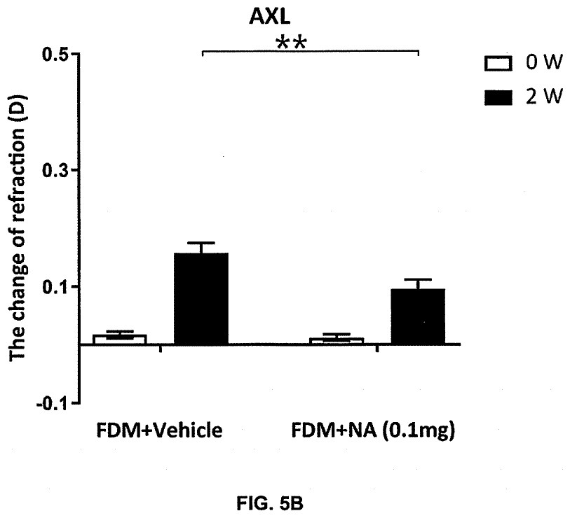

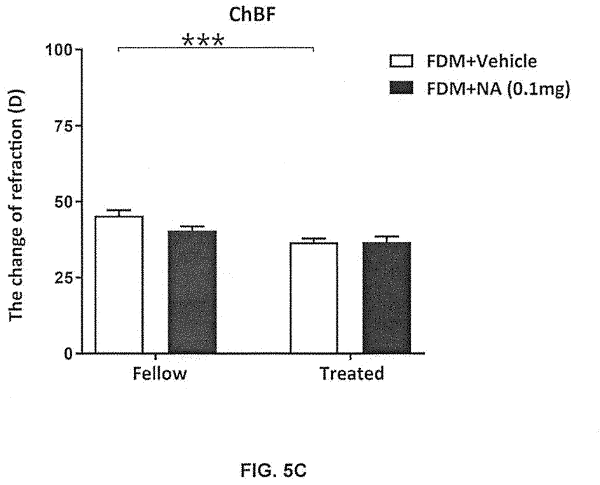

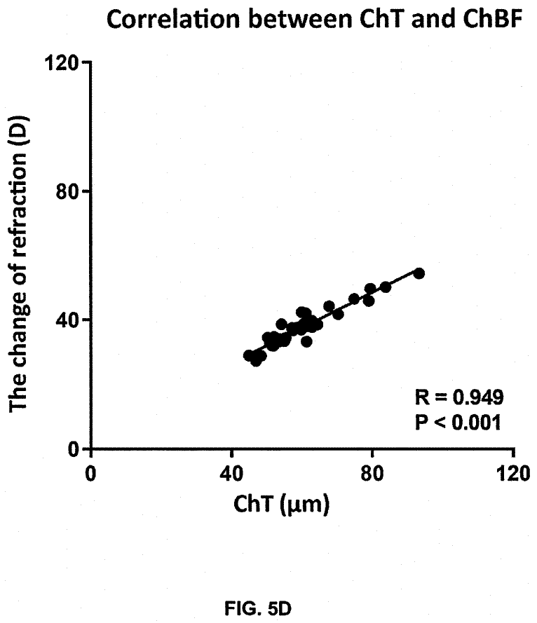

As shown in FIG. 5A-5D, we use a drug that directly dilate a blood vessel or a vascular smooth muscle: nicotinic acid (Niacin, NA), where on 2 weeks after form deprivation an obvious myopia is produced, and 0.1 mg of nicotinic acid can effectively suppress the form deprivation, FDM+Vehicle vs. FDM+NA (0.1 mg): −5.43±1.69 vs. −3.43±1.80 D, p<0.01, FIG. 5A; the eye axis also has the corresponding effect of suppression, 0.17±0.05 vs. 0.10±0.06 mm, p<0.01, FIG. 5B; on 2 weeks after form deprivation the blood flow in the test eye is significantly reduced and statistically significant, fellow eye vs. test eye: 45.38±6.22 vs. 36.62±4.47×103, p<0.001, and after periocular injection of 0.1 mg nicotinic acid, there is no statistical difference between two eyes, FIG. 5C; and there is a high correlation between the choroidal blood flow and the choroidal thickness (R=0.949, p<0.001), as shown in detail in FIG. 5D.

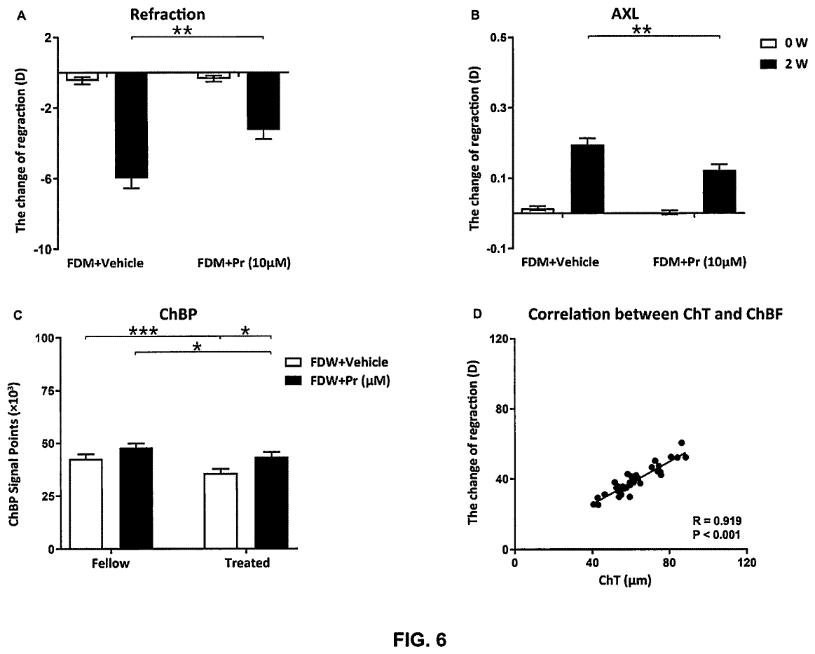

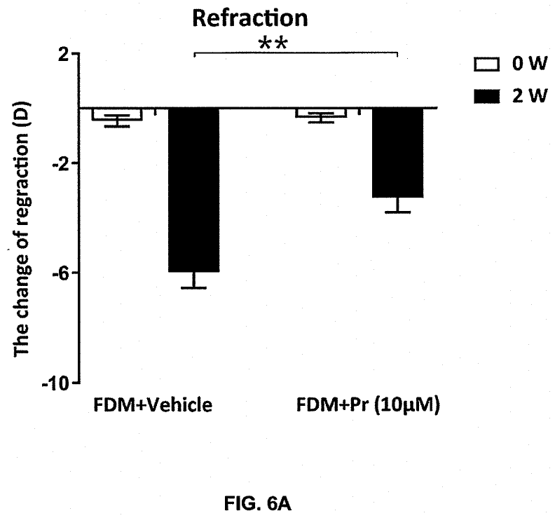

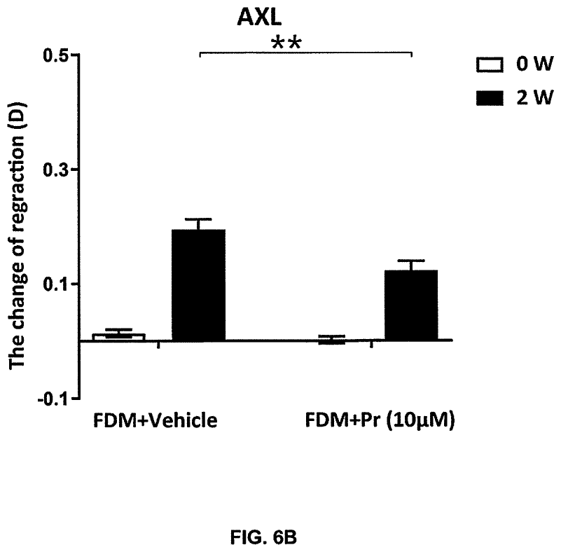

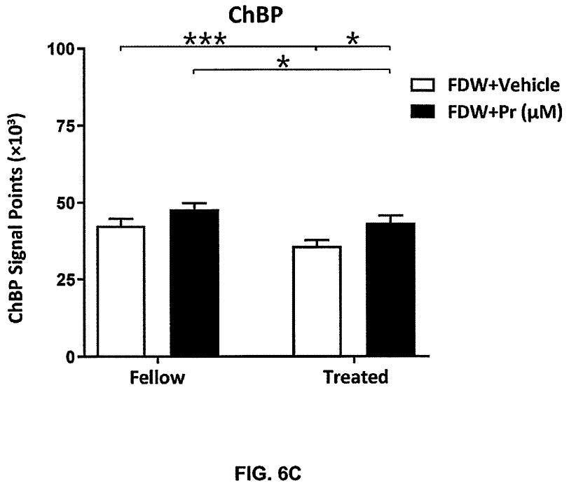

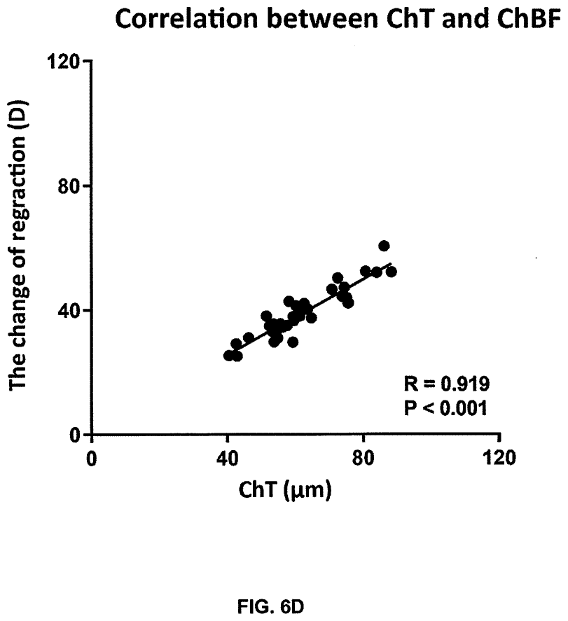

As shown in FIG. 6A-6D, we use an α-adrenergic receptor blocker (α-blocker): prazosin (Prazosin, Pr), where on 2 weeks after form deprivation, an obvious myopia is produced, and 10 μM of prazosin can effectively suppress the form deprivation, F DM+Vehicle vs. FDM+Pr (10 μM): −5.98±2.22 vs. −3.26±2.22 D, p<0.01, FIG. 6A; the eye axis also has the corresponding effect of suppression, 0.17±0.05 vs. 0.10±0.06 mm, p<0.01, FIG. 6B; on 2 weeks after form deprivation, the blood flow of the test eye is significantly reduced and statistically significant, fellow eye vs. test eye: 42.50±8.05 vs. 35.79±6.76×103, p<0.001), and after periocular injection of 0.1 ml of 10 μM prazosin, the reduction of choroidal blood flow is significantly inhibited, FDM+Vehicle vs. FDM+Pr (10 μM): 35.79±6 .76 vs. 37.01±6.26×103, p<0.05, FIG. 6C; and there is a high correlation between the choroidal blood flow and the choroidal thickness (R=0.919, p<0.001), FIG. 6D.

Therefore, we have demonstrated that, the myopia caused by scleral hypoxia can be treated by increasing the volume of choroidal blood flow through expansion of the choroid via a drug that dilates the blood vessels.

Similarly, in the present invention, the myopia caused by scleral hypoxia can also be treated by increasing the volume of choroidal blood flow through expansion of the choroid via a surgical method.

Notices for skilled artisans: Although the present invention has been described in terms of the above specific embodiments, the inventive concept of the present invention is not limited to the invention, and any modification using the inventive concept will be included in the claimed scope of this patent.

The above description is only a preferred embodiment of the present invention, and the claimed scope of the present invention is not limited to the above embodiments, and all the technical solutions under the inventive concept belong to the claimed scope of the present invention. It should be noted that, for those of ordinary skills in the art, several changes and modifications can be made without departing from the principle of the present invention, and these changes and modifications should also be considered as falling within the claimed scope of the present invention.

1. A method for treating myopia, wherein the method is treating myopia caused by scleral hypoxia by increasing the volume of choroidal blood flow.

2. The method for treating myopia according to claim 1, wherein the method comprises increasing the volume of choroidal blood flow by expanding a choroid, so as to achieve treatment of the myopia caused by the scleral hypoxia.

3. The method for treating myopia according to claim 2, wherein the method comprises increasing the volume of choroidal blood flow by expanding the choroid via a medicine, so as to achieve treatment of the myopia caused by the scleral hypoxia.

4. The method for treating myopia according to claim 2, wherein the method comprises, increasing the volume of choroidal blood flow by expanding the choroid via a surgical method, so as to achieve treatment of the myopia caused by the scleral hypoxia.

5. An application of a drug for increasing the volume of choroidal blood flow in preparation of a medicament for treating myopia.

6. The application of the drug for increasing the volume of choroidal blood flow in preparation of a medicament for treating myopia according to claim 5, wherein the drug for increasing the volume of choroidal blood flow is a drug that directly dilates a blood vessel or vascular smooth muscle or a drug that indirectly dilates a blood vessel.

7. The application of the drug for increasing the volume of choroidal blood flow in preparation of a medicament for treating myopia according to claim 6, wherein the drug that directly dilates a blood vessel or vascular smooth muscle is one or more of nicotinic acid, hydralazine, sodium nitroprusside, indapamide, dibazole, papaverine, cinnarizine, merislon or nitroglycerin.

8. The application of the drug for increasing the volume of choroidal blood flow in preparation of a medicament for treating myopia according to claim 6, wherein the drug that indirectly dilates a blood vessel is one or more of a histamine drug, alpha-adrenaline, an angiotensin converting enzyme inhibitor drug, a calcium channel blocker, an anticholinergic agent that blocks a M cholinergic receptor or a traditional Chinese medicine that improves blood circulation.

9. The application of the drug for increasing the volume of choroidal blood flow in preparation of a medicament for treating myopia according to claim 8, wherein the histamine drug is merislon.

10. The application of the drug for increasing the volume of choroidal blood flow in preparation of a medicament for treating myopia according to claim 8, wherein the α-adrenergic receptor blocker is one or more of phentolamine, hydergine or prazosin.

11. The application of the drug for increasing the volume of choroidal blood flow in preparation of a medicament for treating myopia according to claim 8, wherein the angiotensin converting enzyme inhibitor drug is captopril.

12. The application of the drug for increasing the volume of choroidal blood flow in preparation of a medicament for treating myopia according to claim 8, wherein the calcium channel blocker is one or more of nifedipine or verapamil.

13. The application of the drug for increasing the volume of choroidal blood flow in preparation of a medicament for treating myopia according to claim 8, wherein the anticholinergic agent that blocks a M cholinergic receptor is anisodamine.

14. The application of the drug for increasing the volume of choroidal blood flow in preparation of a medicament for treating myopia according to claim 8, wherein the traditional Chinese medicine that improves blood circulation is one or more of ligusticum wallichii, salviae miltiorrhizae, radix pueraiae, a Weierkang tablet, a Chuanshen capsule or Shuxuening.
